(19) United States
(12) Patent Application Publication
(10) Pub. No.: US 2003/0215406 A1
(43) Pub. Date: Nov. 20, 2003

(54) Cosmetic or dermatological preparations containing aminogaunidine, derivatives thereof or structural analogs thereof

(75) Inventors: Volker Schreiner, Hamburg (DE); Thomas Blatt, Hamburg (DE); Melanie Schmidt, Hamburg (DE); Franz Stab, Echem (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/402,028

(22) Filed: Mar. 28, 2003

Related U.S. Application Data
(63) Continuation of application No. PCT/EP01/11039, filed on Sep. 25, 2001.

(30) Foreign Application Priority Data
Sep. 29, 2000 (DE) 100 48 260.0

(51) Int. Cl. A61K 31/715; A61K 7/135; A61K 38/43; A61K 31/4188; A61K 31/385; A61K 31/185; A61K 31/155

(52) U.S. Cl. 424/62; 424/94.1; 514/54; 514/393; 514/440; 514/456; 514/553; 514/634

(57) ABSTRACT

The invention is a cosmetic, dermatological or topical pharmaceutical preparation for use in treating undesired pigmentation of the skin comprising an effective content of one or more of aminoguanidine, derivatives thereof and structural analogs thereof. The invention also includes a method of applying the preparation to affected areas of the skin to treat undesired pigmentation of the skin.

### CROSS-REFERENCE TO RELATED APPLICATIONS

This is a continuation application of PCT/EP01/11039, filed Sep. 25, 2001, which is incorporated herein by reference in its entirety, and also claims the benefit of German Priority Application No.100 48 260.0, filed Sep. 29, 2000.

### FIELD OF THE INVENTION

The present invention relates to cosmetic or dermatological preparations containing aminoguanidine and/or derivatives and structural analogs thereof for cosmetic and topical dermatological skin lightening or for preventing skin tanning, in particular skin tanning caused by UV radiation. In a preferred embodiment, the present invention relates to cosmetic and dermatological preparations for the prophylaxis and treatment of cosmetic or dermatological changes in the skin, such as, for example, undesired pigmentation, for example local hyperpigmentation and incorrect pigmentation (for example liver spots, freckles), or for the purely cosmetic lightening of larger areas of skin which are quite appropriately pigmented for the individual skin type.

### BACKGROUND OF THE INVENTION

Pigmenting of the skin is caused, for example, by melanocytes, which are to be found in the lowest layer of the epidermis, the Stratum basale, alongside the basal cells as pigment-forming cells which, depending on the skin type, occur either individually or in clusters of varying size. Melanocytes contain, as characteristic cell organelles, melanosomes which form melanin to a greater extent when stimulated by UV radiation. This melanin is transported into the keratinocytes and brings about a more or less marked brownish or brown skin color.

Melanin is formed as the end stage of an oxidation process in which tyrosine is finally converted into melanin, under the action of the enzyme tyrosinase, via 3,4-dihydroxyphenylalanine (dopa), dopaquinone, leucodopachrome, dopachrome, 5,6-dihydroxyindole and indole-5,6-quinone.

Problems with skin hyperpigmentation have many different causes and are accompanying phenomena of many biological processes, for example UV radiation (for example freckles, Ephelides), genetic disposition, incorrect pigmentation of the skin during wound healing or scarring or skin aging (for example Lentigines seniles).

Active ingredients and preparations which counteract skin pigmentation are known. In practice, use is made essentially of preparations based on hydroquinone although, on the one hand, these only show their effect after application for several weeks and, on the other hand, application of them for an excessively long time is not always without risk, for toxicological reasons. The inhibition of tyrosinase with substances such as kojic acid, ascorbic acid and azelaic acid and their derivatives is also common, although it has cosmetic and dermatological disadvantages.

### SUMMARY OF THE INVENTION

The object of the present invention was to remedy these shortcomings.

Surprisingly, the object is achieved by cosmetic or pharmaceutical preparations which are characterized by an effective content of aminoguanidine and/or derivatives and structural analogs thereof.

An advantageous embodiment of the present invention is likewise regarded as being the use of aminoguanidine and/or derivatives and structural analogs thereof against undesired pigmentation of the skin and the use of aminoguanidine and/or derivatives and structural analogs thereof for preparing cosmetic or dermatological preparations against undesired pigmentation of the skin.

Aminoguanidine and/or derivatives and structural elements thereof have proven to be excellent active ingredients against undesired pigmentation, both preventatively and also in the sense of a treatment.

According to the invention, the content of aminoguanidine and/or derivatives and structural analogs thereof in the cosmetic or topical dermatological preparations can be 1 ppb (=1 part per billion=10⁻⁷% by weight)−10% by weight, preferably 10 ppb (=0.01 ppm=0.01 part per million=10⁻⁶% by weight)−5% by weight, in particular 0.05 ppm−0.1% by weight, based on the total weight of the preparations.

Surprisingly, it has been found that aminoguanidine and/or derivatives and structural analogs thereof achieve the objects on which the invention is based. By using aminoguanidine and/or derivatives and structural analogs thereof or cosmetic or topical dermatological preparations with an effective content of aminoguanidine and/or derivatives and structural analogs thereof, it is possible to achieve effective prophylaxis against undesired pigmentation. According to the invention, it is, in particular, extremely advantageous to use aminoguanidine and/or derivatives and structural analogs thereof or cosmetic or topical dermatological preparations containing aminoguanidine and/or derivatives and structural analogs thereof for the cosmetic or dermatological treatment of undesired skin pigmentation, i.e., for example, Lentigines seniles.

### DETAILED DESCRIPTION OF THE PREFERRED EMBODIMENTS

The prophylaxis or the cosmetic or dermatological treatment with aminoguanidine and/or derivatives and structural analogs thereof or with the cosmetic or topical dermatological preparations with an effective content of aminoguanidine and/or derivatives and structural analogs thereof takes place in the usual manner, namely by applying aminoguanidine and/or derivatives and structural analogs thereof or the cosmetic or topical dermatological preparations with an effective content of aminoguanidine and/or derivatives and structural analogs thereof to the affected areas of skin.

Aminoguanidine (guanylhydrazine) is characterized by the following structure

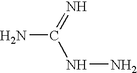

It is a colorless substance which is soluble in water and alcohol, and insoluble in ether. Aminoguanidine forms salts with many acids. It can react as hydrazine, guanidine and formamidine derivatives and can often be converted directly to heterocycles and is used essentially in the form of its sulfate and its hydrogencarbonate.

Aminoguanidine and/or derivatives and structural analogs thereof can advantageously be incorporated into customary cosmetic and dermatological preparations, which can exist in various forms. They can, for example, be a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a solid stick or an aerosol.

For the purposes of the present invention, emulsions according to the invention in the form, for example, of a cream, a lotion or a cosmetic milk, are advantageous and contain, for example, fats, oils, waxes and/or other fatty substances, and water and one or more emulsifiers as are customarily used for this type of formulation.

For the purposes of the present invention, it is also possible and advantageous to add aminoguanidine and/or derivatives and structural analogs thereof to aqueous systems or surfactant preparations for the cleansing of the skin and the hair.

The person skilled in the art is of course aware that high-quality cosmetic compositions are in most cases inconceivable without the customary auxiliaries and additives. These include, for example, bodying agents, fillers, perfume, dyes, emulsifiers, additional active ingredients such as vitamins or proteins, light protection agents, stabilizers, insect repellents, alcohol, water, salts, antimicrobial, proteolytic or keratolytic substances, etc.

Corresponding requirements as to the formulation of medicinal formulations apply mutatis mutandis.

For the purposes of the present invention, medicinal topical compositions generally contain one or more active ingredients in an effective concentration. For the sake of simplicity, in order to distinguish clearly between cosmetic and medicinal use and corresponding products, reference is made to the legal provisions in the Federal Republic of Germany (for example Cosmetics Regulation, Foods and Medicaments Act).

It is also advantageous to add the active ingredient used according to the invention as additive to formulations which already contain other active ingredients for other purposes.

Accordingly, for the purposes of the present invention, cosmetic or topical dermatological compositions can, depending on their composition, be used for example as skin-protection cream, cleansing milk, sunscreen lotion, nutrient cream, day or night cream etc. In some instances, it is possible and advantageous to use the compositions according to the invention as a base for pharmaceutical formulations.

In some instances, those cosmetic and dermatological formulations which exist in the form of a sunscreen are also favourable. In addition to aminoguanidine and/or derivatives and structural analogs thereof, these also preferably contain at least one UVA filter substance and/or at least one UVB filter substance and/or at least one inorganic pigment.

However, it is also advantageous for the purposes of the present invention to provide cosmetic and dermatological preparations whose main purpose is not protection against sunlight, but which nevertheless still comprise a content of UV protection substances. Thus, for example, UV-A and UV-B filter substances are usually incorporated into day creams.

Preparations according to the invention can advantageously comprise substances which absorb UV radiation in the UVB region, the total amount of filter substances for example being from 0.1% by weight to 30% by weight, preferably from 0.5 to 10% by weight, in particular from 1 to 6% by weight, based on the total weight of the preparations.

The UVB filters can be oil-soluble or water-soluble. Examples of oil-soluble substances are:

3-benzylidenecamphor and derivatives thereof, e.g. 3-(4-methylbenzylidene)camphor,

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate; and

2,4,6-trianilino-(p-carbo-2′-ethyl-1′-hexyloxy)-1,3,5-triazine.

Water-soluble Substances are Advantageously:

2-phenylbenzimidazole-5-sulfonic acid and salts thereof, for example sodium, potassium or triethanolammonium salts, sulfonic acid derivatives of benzophenones, preferably 2-hydroxy-4-methoxybenzophenone-5-sulfonic acid and its salts; and

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

The list of given UVB filters which can be used according to the invention is of course not intended to be limiting.

It can also be advantageous to use UVA filters which are usually present in cosmetic and/or dermatological preparations in the preparations according to the invention. Such filter substances are preferably derivatives of dibenzoylmethane, in particular 1-(4′-tert-butylphenyl)-3-(4′-methoxyphenyl)-propane-1,3-dione and 1-phenyl-3-(4′-isopropylphenyl)-propane-1,3-dione. Preparations which comprise these combinations also form part of the subject-matter of the invention. The same amounts of UVA filter substances which were specified for UVB filter substances can be used.

For the purposes of the present invention, cosmetic and/or dermatological preparations can also comprise inorganic pigments which are usually used in cosmetics for protecting the skin against UV radiation. These are oxides of titanium, zinc, iron, zirconium, silicon, manganese, aluminum, cerium and mixtures thereof, and modifications in which the oxides are the active agents. Particular preference is given to pigments based on titanium dioxide. The quantities given for the above combinations can be used.

The cosmetic and dermatological preparations according to the invention can comprise cosmetic active ingredients, auxiliaries and/or additives as are customarily used in such preparations, for example antioxidants, preservatives, bactericides, perfumes, antifoams, dyes, pigments which have a coloring effect, thickeners, surfactants, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

It is likewise advantageous to add conventional antioxidants to the preparations for the purposes of the present invention. According to the invention, favourable antioxidants can be any antioxidants which are customary or suitable for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, βcarotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechols, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin, rutinic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives of said active ingredients which are suitable according to the invention (salts, esters, ethers, sugars, nucleotides, nucleosides, peptides and lipids).

The amount of antioxidants (one or more compounds) in the preparations is preferably from 0.001 to 30% by weight, particularly preferably 0.05-20% by weight, in particular from 1 to 10% by weight, based on the total weight of the formulation.

If vitamin E and/or derivatives thereof are used as the antioxidant(s), it is advantageous to choose their respective concentrations from the range 0.001-10% by weight, based on the total weight of the formulation.

For the purposes of the present invention, if the cosmetic or dermatological formulation is a solution or emulsion or dispersion, the solvent used can be:

water or aqueous solutions

oils, such as triglycerides of capric or caprylic acid, but preferably castor oil;

fats, waxes and other natural and synthetic fatty substances, preferably esters of fatty acids with alcohols of low carbon number, for example with isopropanol, propylene glycol or glycerol, or esters of fatty alcohols with alkanoic acids of low carbon number or with fatty acids; and

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the aforementioned solvents are used. In the case of alcoholic solvents, water can be a further constituent.

The oil phase of the emulsions, oleogels or hydrodispersions or lipodispersions for the purposes of the present invention is advantageously selected from the group of esters of saturated and/or unsaturated, branched and/or unbranched alkanecarboxylic acids having a chain length of from 3 to 30 carbon atoms, and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

In addition, the oil phase can advantageously be selected from the group consisting of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24, in particular 12 to 18 carbon atoms. The fatty acid triglycerides can for example be advantageously selected from the group consisting of synthetic, semi-synthetic and natural oils, e.g. olive oil, sunflower oil, soybean oil, groundnut oil, rapeseed oil, almond oil, palm oil, coconut oil, palm kernel oil and the like.

Any desired mixtures of such oil and wax components can also advantageously be used for the purposes of the present invention. In some instances, it may also be advantageous to use waxes, for example cetyl palmitate, as the sole lipid component of the oil phase.

The oil phase is advantageously selected from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Advantageous hydrocarbons for the purposes of the present invention are paraffin oil, squalane and squalene.

In addition, the oil phase can advantageously contain cyclic or linear silicone oils or consist entirely of such oils, although it is preferred that an additional content of other oil phase components apart from the silicone oil or the silicone oils be used.

Cyclomethicone (octamethylcyclotetrasiloxane) is advantageously employed as the silicone oil to be used according to the invention. However, other silicone oils are also advantageous for the purposes of the present invention, for example hexamethylcyclotrisiloxane, polydimethylsiloxane, poly(methylphenylsiloxane).

In addition, mixtures of cyclomethicone and isotridecyl isononanoate and of cyclomethicone and 2-ethylhexyl isostearate are particularly advantageous.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can be selected advantageously from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

Gels used according to the invention usually comprise alcohols of low carbon number, e.g. ethanol, isopropanol, 1 ,2-propanediol, glycerol and water or an oil mentioned above in the presence of a thickener, which, for oily-alcoholic gels, is preferably silicon dioxide or an aluminum silicate and, for aqueous-alcoholic or alcoholic gels, is preferably a polyacrylate.

Solid sticks comprise, for example, natural or synthetic waxes, fatty alcohols or fatty acid esters. Usual base substances which are suitable for use as cosmetic sticks for the purposes of the present invention are liquid oils (e.g. paraffin oils, castor oil, isopropyl myristate), semisolid constituents (e.g. Vaseline, lanolin), solid constituents (e.g. beeswax, ceresin and microcrystalline waxes and ozokerite) and high-melting waxes (e.g. carnauba wax, candelilla wax).

For the purposes of the present invention, suitable propellants for cosmetic and/or dermatological preparations which can be sprayed from aerosol containers are the customary known, readily volatile, liquefied propellants, for example hydrocarbons (propane, butane, isobutane), which can be used on their own or in mixtures with one another. Compressed air is also advantageous.

The person skilled in the art of course knows that there are propellent gases which are non-toxic per se and would in principle be suitable for realizing the present invention in the form of aerosol preparations but which, because of their harmful effect on the environment or other accompanying circumstances, should be avoided, in particular hydrofluorocarbons and chlorofluorocarbons (CFCs).

For the purposes of the present invention, cosmetic preparations can also be in the form of gels which, in addition to an effective content of aminoquanidine and/or derivatives and structural analogs thereof and solvents which are usually used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel, e.g. in an amount between 0.1 and 30% by weight, preferably between 0.5 and 15% by weight.

The following examples are intended to illustrate the present invention.

### EXAMPLE 1

| W/O cream | % by weight |
|---|---|
| Paraffin oil (DAB 9) | 10.00 |
| Petrolatum | 4.00 |
| Wool wax alcohol | 1.00 |
| PEG-7 Hydrogenated castor oil | 3.00 |
| Aluminum stearate | 0.40 |
| Glycerol | 2.00 |
| Preservatives, dyes, perfume | q.s. |
| Aminoguanidine | 0.2 |
| Water | to 100.00 |

### EXAMPLE 2

| W/O lotion | % by weight |
|---|---|
| Paraffin oil (DAB 9) | 20.00 |
| Petrolatum | 4.00 |
| Glucose sesquiisostearate | 2.00 |
| Aluminum stearate | 0.40 |
| Phytoene | 1.00 |
| Glycerol | 5.00 |
| Preservatives, dyes, perfume | q.s. |
| Aminoguanidine | 2.50 |
| Water | to 100.00 |

### EXAMPLE 3

| O/W lotion | % by weight |
|---|---|
| Paraffin oil (DAB 9) | 8.00 |
| Isopropyl palmitate | 3.00 |
| Petrolatum | 4.00 |
| Cetyistearyl alcohol | 2.00 |
| PEG-40 Castor oil | 0.50 |
| Sodium cetylstearyl sulfate | 0.50 |
| Sodium carbomer | 0.40 |
| Glycerol | 3.00 |
| α-Glucosylrutin | 0.20 |
| Octyl methoxycinnamate | 5.00 |
| Butylmethoxydibenzoylmethane | 1.00 |
| Preservatives, dyes, perfume | q.s. |
| Aminoguanidine | 0.02 |
| Water | to 100.00 |

### EXAMPLE 4

| O/W cream | % by weight |
|---|---|
| Paraffin oil (DAB 9) | 7.00 |
| Avocado oil | 4.00 |
| Glyceryl monostearate | 2.00 |
| Sodium lactate | 3.00 |
| Glycerol | 3.00 |
| Preservatives, dyes, perfume | q.s. |
| Aminoguanidine | 0.40 |
| Water | to 100.00 |

### EXAMPLE 5

| Liposome-containing gel | % by weight |
|---|---|
| Lecithin | 6.00 |
| Shea butter | 3.00 |
| Taurine | 0.20 |
| Sodium citrate | 0.50 |
| Glycine | 0.20 |
| Urea | 0.20 |
| Sodium PCA | 0.50 |
| Hydrolysed collagen | 2.00 |
| Xanthan gum | 1.40 |
| Sorbitol | 3.00 |
| Preservatives, dyes, perfume | q.s. |
| Aminoguanidine | 1.20 |
| Water | to 100.00 |

### EXAMPLE 6

| Sunscreen emulsion | % by weight |
|---|---|
| Cyclomethicone | 2.00 |
| Cetyldimethicone copolyol | 0.20 |
| PEG-22 dodecyl copolymer | 3.00 |
| Paraffin oil (DAB 9) | 2.00 |
| Caprylic/capric triglyceride | 5.80 |
| Octyl methoxycinnamate | 5.80 |
| Butylmethoxydibenzoylmethane | 4.00 |
| Aminoguanidine | 0.50 |
| D-Biotin | 0.50 |
| ZnSO₄ | 0.70 |
| Na₄EDTA | 0.30 |
| Preservatives, dyes, perfume | q.s. |
| Water | to 100.00 |

### EXAMPLE 7

| Sunscreen emulsion | % by weight |
|---|---|
| Cyclomethicone | 2.00 |
| Cetyl stearyl alcohol + PEG-40 hydrogenated | 2.50 |
| castor oil + sodium cetyistearyl sulfate | |
| Glyceryl lanolate | 1.00 |
| Caprylic/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropyl methylcellulose | 0.30 |
| Octyl methoxycinnamate | 5.00 |
| Butylmethoxydibenzoylmethane | 0.50 |
| Aminoguanidine | 0.20 |
| α-Tocopheryl acetate | 1.00 |
| Na₃HEDTA | 1.50 |
| Preservatives, dyes, perfume | q.s. |
| Water | to 100.00 |

### EXAMPLE 8

| Sunscreen emulsion | % by weight |
|---|---|
| Cyclomethicone | 2.00 |
| Cetylstearyl alcohol + PEG-40 hydrogenated | 2.50 |
| castor oil + sodium cetyistearyl sulfate | |
| Glyceryl lanolate | 1.00 |
| Caprylic/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropylmethylcellulose | 0.30 |
| Octyl methoxycinnamate | 5.00 |
| Butylmethoxydibenzoyimethane | 0.75 |
| Na₃HEDTA | 1.50 |
| Preservatives, dyes, perfume | q.s. |
| Aminoguanidine | 3.00 |
| Water | to 100.00 |

### EXAMPLE 9

| Spray formulation | % by weight |
|---|---|
| Ubiquinone 10 | 0.10 |
| Aminoguanidine | 0.80 |
| Ethanol | 28.20 |
| Preservatives, dyes, perfume | q.s. |
| Propane/butane 25/75 | to 100.00 |

### EXAMPLE 10

| O/W Creme | % by weight |
|---|---|
| Paraffin oil (DAB 9) | 7.00 |
| Soybean oil | 4.00 |
| Glyceryl monostearate | 2.00 |
| Sodium lactate | 3.00 |
| Glycerol | 8.00 |
| Preservatives, dyes, perfume | q.s. |
| Aminoguanidine | 0.08 |
| Water | to 100.00 |

That which is claimed:

1. A cosmetic, dermatological or topical pharmaceutical preparation for treating undesired pigmentation of the skin, comprising an effective amount of one or more of aminoguanidine, derivatives thereof, and structural analogs thereof.

2. The preparation as claimed in claim 1, wherein the aminoguanidine, derivatives thereof and structural analogs thereof are present in concentrations of from 1 part per billion by weight (10⁻⁷% by weight) to 30% by weight, based on the total weight of the preparation.

3. The preparation as claimed in claim 1, wherein the aminoguanidine, derivatives thereof and structural analogs thereof are present in concentrations of from 10 parts per billion by weight (10⁻⁶% by weight) to 5% by weight, based on the total weight of the preparation.

4. The preparation as claimed in claim 1, wherein the aminoguanidine, derivatives thereof and structural analogs thereof are present in concentrations of from 0.05 parts per billion by weight to 0.5% by weight, based on the total weight of the preparation.

5. The preparation as claimed in claim 1, further comprising one or more additional active ingredients or derivatives thereof selected from the group consisting of alpha-lipoic acid, phytoene, D-biotin, coenzyme Q10, alpha-glucosylrutin, creatine, carnitine, carnosine, isoflavone and taurine.

6. The preparation as claimed in claim 5, wherein the additional active ingredients or derivatives thereof are present in a content of from 0.0001 to 30% by weight, based on the total weight of the preparation.

7. The method of treating undesired pigmentation of the skin by applying to affected areas of the skin a cosmetic, dermatological or topical pharmaceutical preparation comprising an effective amount of one or more of aminoguanidine, derivatives thereof and structural analogs thereof.

8. The method as claimed in claim 7, wherein the aminoguanidine, derivatives thereof and structural analogs thereof are present in concentrations of from 1 part per billion by weight (10⁻⁷% by weight) to 30% by weight, based on the total weight of the preparation.

9. The method as claimed in claim 7, wherein the aminoguanidine, derivatives thereof and structural analogs thereof are present in concentrations of from 10 parts per billion by weight (10⁻⁶% by weight) to 5% by weight, based on the total weight of the preparation.

10. The method as claimed in claim 7, wherein the aminoguanidine, derivatives thereof and structural analogs thereof are present in concentrations of from 0.05 parts per billion by weight to 0.5% by weight, based on the total weight of the preparation.

11. The method as claimed in claim 7, further comprising one or more additional active ingredients or derivatives thereof selected from the group consisting of alpha-lipoic acid, phytoene, D-biotin, coenzyme Q10, alpha-glucosylrutin, creatine, carnitine, carnosine, isoflavone and taurine.

12. The method as claimed in claim 11, wherein the additional active ingredients or derivatives thereof are present in a content of from 0.0001 to 30% by weight, based on the total weight of the preparation.

13. The method as claimed in claim 7, wherein the method comprises applying the preparation to areas of the skin affected by Lentigines seniles.
